(19) United States
(12) Patent Application Publication
(10) Pub. No.: US 2012/0087900 A1
(43) Pub. Date: Apr. 12, 2012

(54) IMMUNOTHERAPEUTIC AGENT

(75) Inventor: Tai-Gyu KIM, Seoul (KR)

(73) Assignee: CATHOLIC UNIVERSITY INDUSTRY ACADEMIC COOPERATION FOUNDATION, Seoul (KR)

(21) Appl. No.: 13/078,836

(22) Filed: Apr. 1, 2011

(30) Foreign Application Priority Data
Oct. 7, 2010 (KR) 10-2010-0097991

(51) Int. Cl. A61K 35/14; C12N 15/85; A61P 31/12; C12N 15/88; C12P 21/00; A61K 35/00; C12N 5/10; C12N 15/87

(52) U.S. Cl. 424/93.21; 435/325; 435/455; 435/461; 435/458; 435/69.52

(57) ABSTRACT

The present invention is directed to adoptive immunotherapy using a lymphocyte in which an antigen-specific receptor and a bioactive material gene such as an IL-2 gene or a water-soluble TGF-beta receptor gene are transferred. The bioactive material is intensively secreted to, for example, a local site of a tumor, thereby reducing systemic side effects as much as possible, and the survival time of the lymphocyte is increased, thereby further improving the effect of the adoptive immunotherapy.

### CROSS-REFERENCE TO RELATED APPLICATION

This application claims priority to and the benefit of Korean Patent Application No. 2010-0097991, filed Oct. 7, 2010, the disclosure of which is incorporated herein by reference in its entirety.

### BACKGROUND

1. Field of the Invention

The present invention relates to an immunotherapeutic agent comprising a lymphocyte having an antigen-specific receptor gene and a bioactive material gene. The antigen-specific lymphocyte can penetrate into, for example, a specific tumor site, and can locally transfer an immune factor such as IL-2 to increase the penetration of NK cells into a tumor and proliferate without an external treatment with an immune factor, thereby providing excellent lymphocyte viability and immune response induction effect.

2. Discussion of Related Art

Successful clinical results of T cell adoptive immunotherapy support the medical importance of future treatment of antitumor cells. Generally, adoptive immunotherapy is a method of sampling blood from a patient, producing antigen-specific T cells, and injecting the T cells into the patient. While tumor immunotherapy targeting tumor antigens has been actively researched and developed, it still has many problems in being medically commercialized.

For clinical application, since reproducibility in production of antigen-specific T cells against a tumor is low due to individual differences, and the frequency of antigen-specific T cells is low as 1% or so of the total cells even when the antigen-specific T cells are successfully produced in vitro, safety regarding in vivo side effects caused by T cells occupying 99% of the total cells should be considered.

As a new approach to generalize immunotherapy using T cells, adoptive immunotherapy through the transfer of a T cell receptor (TCR) is being developed. However, since the antigen-specific TCR has not been clearly investigated, a method using a chimeric immune receptor (CIR) into which a tumor antigen-specific single chain antibody (scFv) and CD28 and CD3ζ, which are signal transduction sites of the TCR, are inserted is being researched.

For receptor gene transfer, a retrovirus has been mainly used, but it has emerging problems such as low transfer rate, time, costs to produce viruses, limited number of insertable genes, and safety for clinical research. Recently, there has been in vitro research reporting efficiency of the TCR RNA transfer to replace DNA, and research in which temporal expression of RNA also exhibits the same antitumor response in vivo as DNA expression has been progressing.

Meanwhile, IL-2 is a representative material in cytokine gene therapy, and its effect has been proven by much research. However, according to systemic IL-2 therapy, depending on the reaction site, side effects including capillary leak syndrome have become problems. In addition, it has been reported that, compared to the amount of IL-2 used in the therapy, local IL-2 therapy is more effective than the systemic IL-2 therapy.

### SUMMARY OF THE INVENTION

The present invention is directed to adoptive immunotherapy using a lymphocyte in which an antigen-specific receptor and a bioactive material gene such as an IL-2 gene or a water-soluble TGF-beta receptor gene are transferred. The bioactive material is intensively secreted to a local site of a tumor, thereby reducing systemic side effects as much as possible, and the survival time of the lymphocyte is increased, thereby further improving the effect of the adoptive immunotherapy.

The present invention resolves the conventional problems by transferring an antigen-specific receptor and bioactive material gene such as an IL-2 gene or a water-soluble TGF-beta receptor to a lymphocyte, and injecting the lymphocyte into a tumor. More particularly, the present invention provides an immunotherapeutic agent having excellent lymphocyte viability and immune response induction. The lymphocyte expressing an anti-Her-2/neu-specific CIR and a bioactive material prepared using DNA or RNA transfer may enhance an in vivo antitumor effect of a CIR in response to the tumor-specific antigen in the tumor site because of local secretion of the bioactive material.

### BRIEF DESCRIPTION OF THE DRAWINGS

The above and other objects, features and advantages of the present invention will become more apparent to those of ordinary skill in the art by describing in detail exemplary embodiments thereof with reference to the adhered drawings, in which:

FIG. 1 shows CIR and IL-2 expression in an activated human peripheral blood lymphocyte by RNA electroporation: a) shows CIR expression obtained from mock-PBL, IL-2-PBL, CIR-PBL and CIR/IL-2-PBL a day after RNA transfer, b) shows CIR expression obtained from CIR/IL-2-PBL and mock-PBL every day after RNA transfer, c) shows ELISA results for IL-2 secretion in supernatants obtained from mock-PBL, IL-2-PBL, CIR-PBL and CIR/IL-2-PBL a day after RNA transfer, d) shows IL-2 secretion in supernatants obtained from CIR/IL-2-PBL every day after RNA transfer, and e) shows IFN-γ secretion in mock-PBL, IL-2-PBL, CIR-PBL and CIR/IL-2-PBL co-incubated with SKOV3 cells;

FIG. 2 shows the survival time of a lymphocyte into which CIR and/or IL-2 RNA are transferred in vitro: a) shows absolute cell counts of mock-PBL, IL-2-PBL, CIR-PBL and CIR/IL-2-PBL, which are incubated in the absence of IL-2, and b) shows a cell count of annexin V and propidium iodide (P1)-positive cells;

FIG. 3 shows viability of lymphocytes in the spleen and tumor in an animal model injecting a lymphocyte to which RNAs of a tumor-specific CIR and IL-2 are transferred, which is measured by carboxyfluorescein succinimidyl ester (CFSE) staining analysis;

FIG. 4 shows inhibition of tumor growth in each group after RNAs of a tumor-specific CIR and IL-2 are simultaneously or individually injected into an animal model;

FIG. 5 shows a number of natural killer (NK) cells present in a tumor site (a) and a spleen site (b) after a lymphocyte to which RNAs of a tumor-specific CIR and IL-2 are transferred is injected into an animal cell; and

FIG. 6 shows comparison of an increase in tumor volume between a control group (a) and a group (b) in which NK cells are removed from an animal model and then a lymphocyte is injected, before the lymphocyte to which RNAs of a tumor-specific CIR and IL-2 are transferred is injected into the animal model.

### DETAILED DESCRIPTION OF EXEMPLARY EMBODIMENTS

Hereinafter, the present invention will be described with reference to examples and comparative examples in detail. However, the present invention is not limited to these examples.

The present invention relates to an immunotherapeutic agent comprising a lymphocyte having an antigen-specific receptor gene and a bioactive material gene as an effective component.

In the present invention, the antigen-specific receptor may be, but is not limited to, a tumor antigen-specific receptor and an antigen-specific T cell receptor, and preferably, an anti-Her-2/neu-specific CIR.

In the present invention, the bioactive material gene may be, but is not limited to, an IL-2 gene or a TGF-beta receptor gene.

In the present invention, the bioactive material gene may be IL-2 RNA, and the RNA may be transferred to the lymphocyte by electroporation or via liposomes.

In the present invention, the lymphocyte may be a cell derived from a lymphocyte of peripheral blood, which is a T lymphocyte activated in a short term, a T-lymphocyte expanded on a large scale in vitro, etc. (ex. CD4+ T cell, CD8+ T cell, gamma delta T cell, NK cell, cytokine induced killer cell(CIR), etc.).

The present invention provides an anticancer composition comprising a lymphocyte to which RNAs of a tumor-specific CIR and a bioactive material are transferred, as an effective component. In particular, the tumor-specific CIR RNA which may be transferred to the lymphocyte may include, but is not limited to, RNA of an anti-Her-2/neu CIR.

The immunotherapeutic agent of the present invention can be applied in various cancer therapies, immunization, and viral disease therapies according to a kind of introduced antigen-specific receptors. Specifically, the immunotherapeutic agent can prevent or treat cancers overexpressing Her-2/neu, for example, ovarian cancer. Also, the immunotherapeutic agent can prevent or treat viral diseases occurred under immunosuppression state. For example, Epstein-Barr Virus and Cytomegalovirus may causes infectious diseases in patients who are received with hematopoietic stem cell transplantations and immunosuppressant. Such viral diseases occurred after transplantation can be prevented or treated by the immunotherapeutic agent of the present invention if viral antigen-specific receptor is used as an antigen specific receptor of the present invention.

Particularly, the present invention provides a composition for preventing or treating cancer comprising a lymphocyte to which RNAs of an anti-Her-2/neu-specific CIR and IL-2 are transferred as an effective component.

The present invention provides a method of preparing a lymphocyte comprising providing an antigen-specific receptor gene and a bioactive material gene, and transferring the genes to a lymphocyte.

The present invention also provides a method of preparing an anticancer therapeutic agent comprising preparing a lymphocyte to which RNAs of a tumor-specific CIR and a bioactive material are introduced.

In further detail, the present invention provides a method of preparing an anticancer therapeutic agent comprising providing RNAs of a tumor-specific CIR and a bioactive material, and transferring the RNAs to a lymphocyte.

The tumor-specific CIR may be an anti-Her-2/neu CIR.

The bioactive material gene may be an IL-2 gene or a TGF-beta receptor gene.

In the present invention, the RNAs may be transferred by electroporation or via liposomes.

The present invention also provides a method of preventing or treating a cancer or a viral disease comprising administrating a therapeutically effective amount of lymphocyte to which RNAs of an antigen-specific receptor and a bioactive material are introduced to a mammal.

The present invention provides a method of preventing or treating cancer comprising administrating a therapeutically effective amount of lymphocytes to which RNAs of an antigen-specific receptor and a bioactive material are transferred to a mammal having a tumor, thereby inhibiting growth of the tumor.

The present invention also provides a method of locally expressing a bioactive material in an antigen-specific lymphocyte-specific site using a lymphocyte to which an antigen-specific receptor gene and a bioactive material gene are transferred. For example, when an IL-2 gene or a TGF-beta receptor gene is used as the bioactive material gene, the survival time of the lymphocyte is increased, and the bioactive material may be locally applied to the antigen-specific lymphocyte-specific site.

The anticancer composition comprising the lymphocyte to which RNAs of a tumor-specific CIR and a bioactive material are introduced according to the present invention can minimize systemic side effects due to a tumor site-specific antitumor effect, prolong an anticancer effect due to extended survival of the lymphocyte, and induce an immune response, resulting in both prevention and treatment of cancer.

The anticancer composition comprising the lymphocyte to which RNAs of a tumor-specific CIR and a bioactive material are introduced according to the present invention may be administered parenterally, for example, by intravascular injection. The administration may be performed with a single or multiple dose schedules. The anticancer composition may be administered with other immunomodulators. Such an anticancer composition comprises the lymphocyte of the present invention together with at least one pharmaceutically acceptable carrier and/or diluent. Here, such a carrier may include any carriers which cause no harmful reaction in an individual to which the composition is administered.

The composition of the present invention may usually include a diluent, for example, water, saline, glycerol, or ethanol, and an excipient, for example, a wetting agent, an emulsifier, or a pH buffer may be present in the composition. Suitable types for injection include a sterilized water-soluble solution or dispersion and sterilized powder for instantly preparing a sterilized injectable solution or dispersion. These materials need to be stabilized under their preparing conditions, and preserved against contamination by microorganisms such as bacteria or fungi. The contamination by the microorganism may be prevented by using various antibacterial agents and antifungal agents, for example, paraben, chlorobutanol, phenol, sorbic acid, and thimerosal. In many cases, an isotonic agent, for example, saccharide or sodium chloride may be included. Long-time absorption of the injectable composition may be achieved by using a composition containing an absorption-delaying agent, for example, aluminum monostearate or gelatin. A sterilized injectable solution may be prepared by binding a required amount of lymphocytes to various other components mentioned above in the above-mentioned solvent, and performing sterile-filtration of the other components excluding the lymphocyte, for example, PBS buffer. Generally, dispersion may be prepared by diffusing various sterilized active components into a sterilized vehicle containing other required components from a basic dispersion medium and the above-mentioned solution. For example, the sterilized powder for preparing a sterilized injectable solution may be prepared by vacuum drying and freeze-drying techniques. By such a technique, powder composed of the desired components may be obtained from an active component and the sterile-filtrated solution thereof.

The delivery of the lymphocyte according to the present invention is useful to induce an immune response without limitation to the reaction of the present invention in any way. Specifically, it is useful to induce the immune response by NK cells or the response of a cytotoxic T lymphocyte against an antigen. The immune response may be a specific (T cell- and/or B cell-specific) and/or non-specific immune response. Therefore, another aspect of the present invention relates to a method of causing, inducing or stimulating an immune response against an antigen in a human, specifically, a cancer patient. Such method comprises administrating a therapeutically effective amount of the above-mentioned cancer therapeutic composition or lymphocyte to a cancer patient.

The immune response may include an immune response by NK cells, and a cytotoxic T lymphocyte response. The cytotoxic T lymphocyte response may occur in combination or alone with a helper T cell response, a humoral response or other specific or non-specific immune responses.

The administration of the composition of the present invention causes, induces, or otherwise stimulates an immune response to inhibit, suspend, delay or prevent the occurrence or development of diseases.

Generally, the composition is directly delivered by subcutaneous, abdominal, intravascular or intramuscular injection, or delivered to a gap between tissues. The composition may also be administered to a lesion. The administration may be performed on a single or multiple dose schedule.

The term “therapeutically effective amount” is an amount required to at least partially induce a desired immune response or to delay or completely terminate the occurrence or development of a certain disease to be treated. Such an amount is varied depending on the health and physical state of the individual to be treated, a taxonomic group of the individual to be treated, ability of the immune system of an individual synthesizing an antibody, a desired degree of protection, an immunotherapeutic formulation, evaluation on medical conditions, and other related factors. It is expected that such an amount is included in a relatively wide range of measurables by common trials, and for example, to treat ovarian cancer, with respect to a 60 kg adult, a composition may be administered at 2×10⁶ to 2×10⁷ cells/injection per dosage.

Hereinafter, the present invention will be described with reference to the examples according to the present invention and comparative examples not according to the present invention in further detail, but the scope of the present invention is not limited to the following examples.

### EXAMPLES

### Materials and Methods

Cells and Antibodies

Human PBLs were grown in RPMI 1640 medium supplemented with 10% (v/v) fetal bovine serum (FBS), 2 mM glutamine, penicillin (100 U/ml) and streptomycin (10 μg/ml) (Invitrogen, Gibco, Grand Island, N.Y.). The human ovarian cancer cell line SKOV3, which expresses the Her-2/Neu antigen, was maintained in Dulbecco's modified Eagle's medium with the following additives: 10% (v/v) FBS, 2 mM glutamine, penicillin (100 U/ml) and streptomycin (100 μg/ml) (Invitrogen Gibco). The following mAbs were used in this study: 9E10 (mouse anti-cMyc FITC conjugated; Sigma, St Louis, Mo.), mouse anti-mouse Pan-NK (CD49b) PE-conjugated (eBioscience, San Diego, Calif.), anti-CD3 mAb (OKT3; Ortho Biotech Inc., Raritan, N.J.).

In Vitro Transcription of Messenger RNA

The CIR containing anti-Her-2/neu scFv (single-chain variable fragment) joined to tag sequence for detection from c-myc region, the intracellular portion of CD28 and CD3ζ was kindly provided by Philip K Darcy (Peter MacCallum Cancer Centre, Australia). The CIR construction was subcloned into the pcDNA3.1 vector (Invitrogen) with the T7 promoter proximal to the start codon of the backbone. The pcDNA3.1-IL-2 is an expression vector combining human IL-2 cDNA. The in vitro transcription was performed with T7 RNA polymerase using the mMESSAGE mMACHINE T7 Ultra kit (Ambion Inc., Austin, Tex.) according to the manufacturer's instructions. After in vitro transcription, the mRNA was purified using phenol/chloroform (Sigma) and purified RNA was eluted in RNase-free water at 4-5 mg/ml.

RNA Electroporation of Primary Human PBLs

Transduction of human PBLs was performed as described previously. Briefly, peripheral blood mononuclear cells from healthy donors were isolated by Ficoll-Paque density centrifugation (GE Healthcare Bio-Sciences, Uppsala, Sweden). PBLs were isolated from human peripheral blood mononuclear cells by depleting CD14⁺ cells using microbeads (Miltenyi, Auburn, Calif.). CDI4⁻ PBLs were activated with anti-CD3 (ORTHOCLONE OKT3, 500 ng/ml; Ortho Biotech, Bridgewater, N.J.) and recombinant human IL-2 (600 IU/ml; eBioscience) for 3 days. Stimulated PBLs were collected and resuspended at 1×10⁶ cells per 100 μl in OPTI-MEM (Invitrogen Gibco) containing mRNA. Cells were electroporated in 2-mm cuvettes at 400V for 500 ms, using a square waveform generator (ECM 830 Electro Square Porator; BTX, a division of Genetronics, San Diego, Calif.). The electroporated PBLs were immediately transferred to medium containing 10% FBS cultured in 24-well tissue culture plates (2 ml per well). The next day, cell were harvested and used for flow cytometry analysis or functional assay. PBLs were grown in RPMI 1640 medium supplemented with 10% (v/v) FBS, 2 mM glutamine, penicillin (100 U/ml) and streptomycin (100 μg/ml; Invitrogen Gibco).

Cytokine Measurement

Cytokine secretion by transduced PBLs on encountering target antigen was detected by enzyme-linked immunosorbent assay (eBioscience). Briefly, each of the RNA-transduced PBLs (1×10⁶ cells per ml) (CIR/IL-2-PBL, CIR-electroporated PBL (CIR-PBL), IL-2-PBL, mock-PBL) was incubated with Her-2/Neu-positive cells, SKOV3, in 48-well culture plates for 6 days at 37° C. Each day, supernatants were collected and the levels of expression of IFN-γ and IL-2 RNA were determined.

Evaluation of Apoptosis

Apoptosis was detected by dual staining with FITC-conjugated Annexin V and propidium iodide (PI; BD Pharmingen, San Diego, Calif.). Transduced PBLs were divided into 24 wells and cultured for 6 days. Each day, cells from one of the 24 wells were collected and stained for apoptosis.

CFSE Labeling for In Vivo Tracking

RNA-transduced PBLs were incubated with carboxyfluorescein succinimidyl ester (CFSE; Molecular Probes, Eugene, Oreg.) at a final concentration of 5 mM for 10 min at 37° C. The reaction was stopped by washing with ice-cold phosphate-buffered saline (PBS) supplemented with 10% FBS. CFSE-labeled RNA-transduced PBLs (2×10⁶) were administered into mice by intra-tumoral injection (Balb/c nu/nu; Orient Bio, Gyeonggi do, Korea). On a day after injection, the spleen and tumor were removed, minced and passed through a nylon mesh to yield a single-cell suspension. Erythrocytes were lysed by re-suspending the cells in an ACK lysis buffer and immediately placing on ice for 10 min. The cells were washed with PBS and immediately fixed in paraformaldehyde (1% final concentration). The cells were detected by flow cytometry.

Nude Mouse Xenograft Model and Depletion of NK Cells

Female athymic 4-week-old nude mice (Balb/c nu/nu; Orient Bio) were housed under specific pathogen-free conditions according to the guidelines of the animal care committee. All mice were ear-tagged and their individual tumor volumes were monitored during the experiment. After 1 week of acclimatization, mice were inoculated subcutaneously (26-gauge needle) with 1×10⁷ SKOV3 cells in a 0.4-ml single-cell suspension. After tumors become palpable, the antitumor efficacy of the intra-tumoral injection of 2×10⁶ PBLs expressing CIR and IL-2 was assessed and compared with that of CIR, IL-2 mock-PBLs and PBS. For depletion of NK cells, we intravenously administered 50 μl of antiasialo-GM1 antibody (Waco Pure Chemical Industries, Osaka, Japan) 6 days before tumor cell injection and once weekly thereafter, for a total of four injections. The treatments were performed thrice at 5-day intervals and mice were monitored daily for tumor growth. Following treatment, the tumor volume (mm³) was estimated in accordance with the formula (tumor width² (mm)×tumor length (mm)/2). All animals were handled according to the local and national regulations, and the local ethics committee approved the protocol.

Statistics

Statistical analysis was performed using the two-tailed Student's t-test for cytokine assay and the two-tailed Mann-Whitney nonparametric test for tumor volume measurement.

Results

Expression and Function of CIR and IL-2

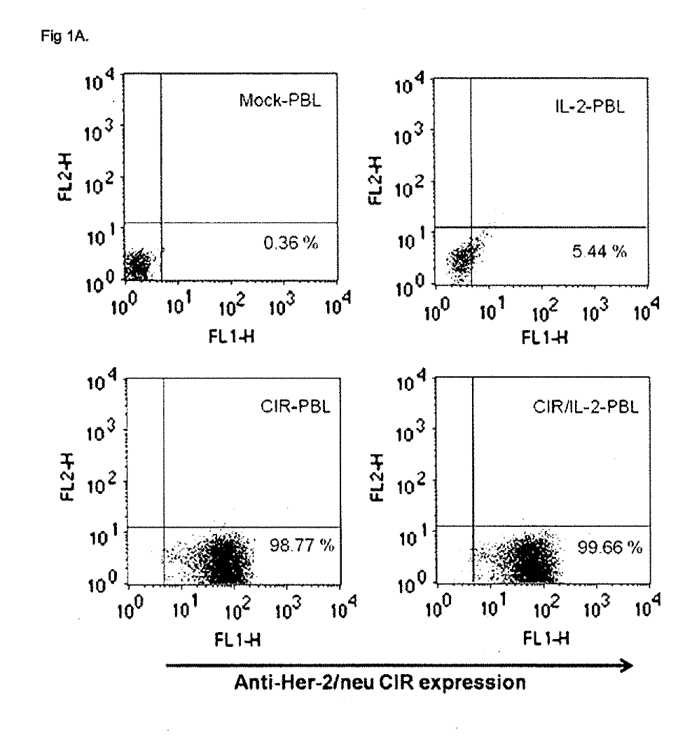

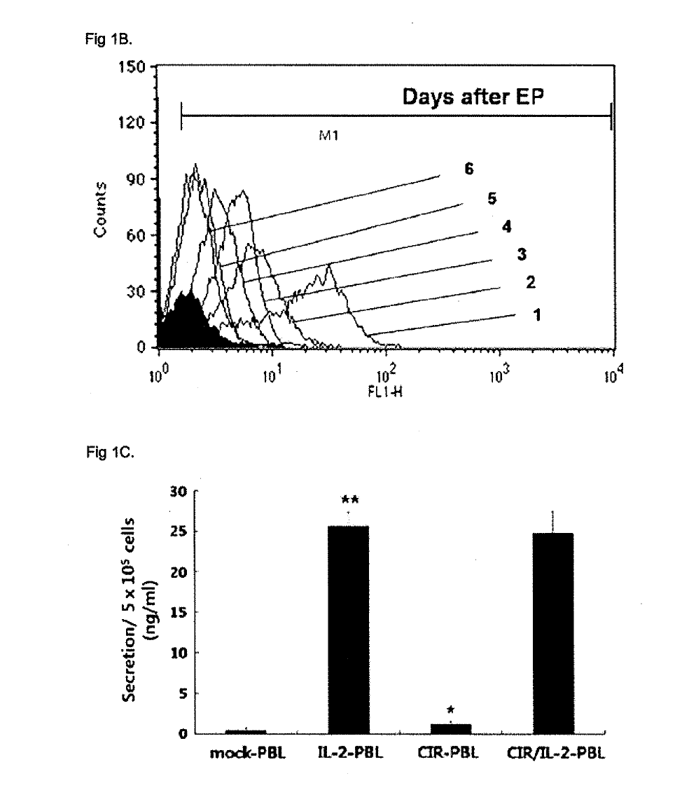

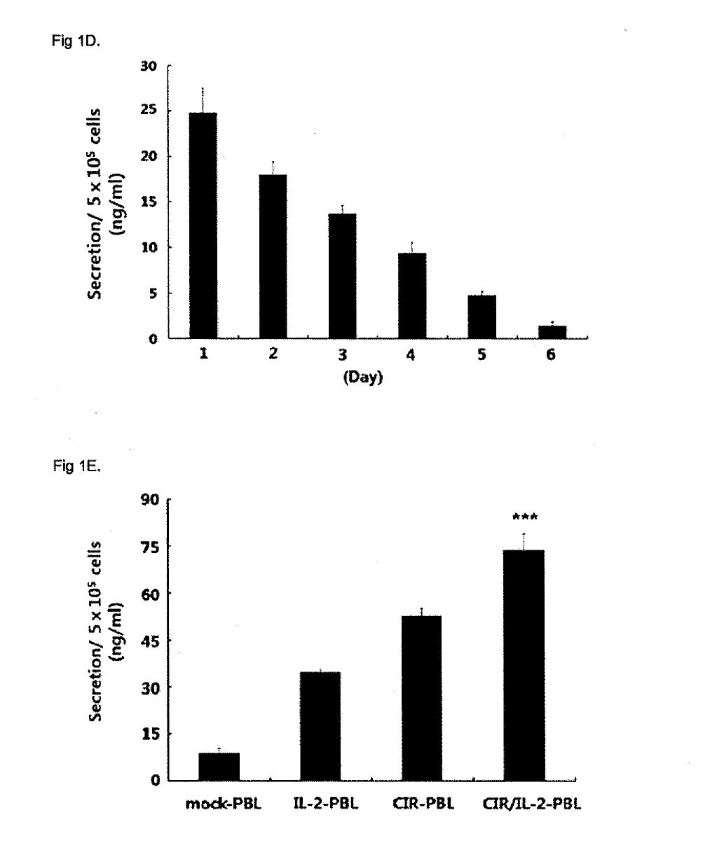

After 3 days of stimulation with OKT3 and IL-2, we electroporated human PBLs were RNA encoding CIR and/or IL-2 against Her-2/neu antigen. CIR was highly expressed in more than 98% of cells electroporated with CIR RNA or with CIR and IL-2 (CIR/IL-2) RNA on day 1 (FIG. 1a). There was no expression of CIR in mock-PBL or in IL-2-PBL. We also tested the kinetics of CIR expression with CIR/IL-2-PBL (FIG. 1b). Electroporation with 10 μg of RNA resulted in mean fluorescence intensity of about 76.9 on day 1. On day 2, the expression intensity was decreased to about one-third. On day 3, the mean fluorescence intensity decreased again to one-third of that observed the day before. After day 3, the mean fluorescence intensity remained at about 6-9 until day 6.IL-2-PBL, and CIR/IL-2-PBL highly expressed IL-2 (25.7±1.7 and 24.8±2.8 ng/ml, **P=0.28) on day 1 after electroporation (FIG. 1c). There was very low secretion of IL-2 in mock-PBL and CIR-PBL. FIG. 1d shows IL-2 secretion by CIR/IL-2-PBL. A high level of IL-2 production (24.8±2.8 ng/ml) occurred on day LAs observed by flow cytometry for CIR expression, secretion potency of IL-2 gradually decreased until day 6 (1.4±0.5 ng/ml). To define the antigen-specific response of CIR to Her-2/neu, we measured IFN-γ production on day 1 on stimulation with SKOV3 cells (FIG. 1e). CIR-PBL and CIR/IL-2-PBL showed higher expression of IFN-γ (52.8±2.4 and 73.8±5.4 ng/ml) than did mock-PBL (8.8±1.6 ng/ml). IL-2-PBL also secreted intermediate levels of IFN-γ (34.8±0.9 ng/ml). These results show that, after RNA electroporation into activated PBLs, both CIR and IL-2 were successfully expressed and functionally active.

Prolonged Survival of PIM by Transfer of IL-2 RNA

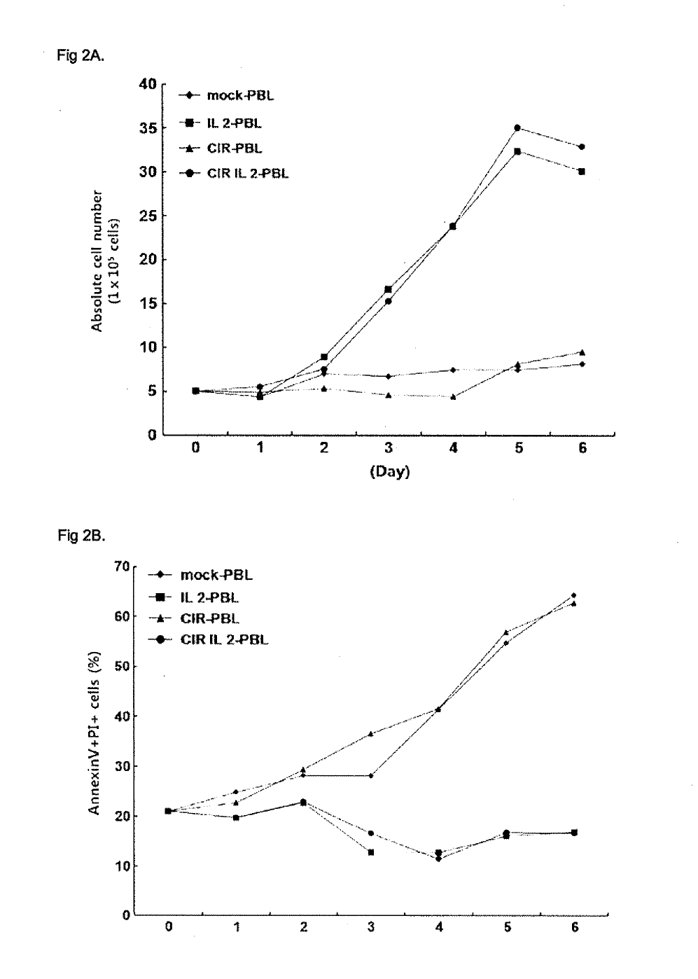

After RNA electroporation, the number of PBLs in culture did not differ significantly among groups until day 2. The number of mock-PBL and CIR-PBL cells remained similar to the number present immediately after electroporation, whereas the number of IL-2-PBL and CIR/IL-2-PBL increased gradually after day 2 (6.5- and 7-fold at day 5, respectively; FIG. 2a). In the apoptosis assay using Annexin V and PI staining (FIG. 2b), the percentage of apoptotic cells in mock-PBL and CIR-PBL gradually increased from about 21% on day 0 to more than 65% after day 5. However, the percentage of apoptotic IL-2-PBL and CIR/IL-2-PBL did not increase at all. The effects of IL-2 RNA transfer were similar to those observed when exogenous IL-2 was added in culture (data not shown). These results suggest that transfer of IL-2 RNA can induce proliferation and prolong survival of activated PBLs while inhibiting apoptosis.

Migration of Infused PBLs

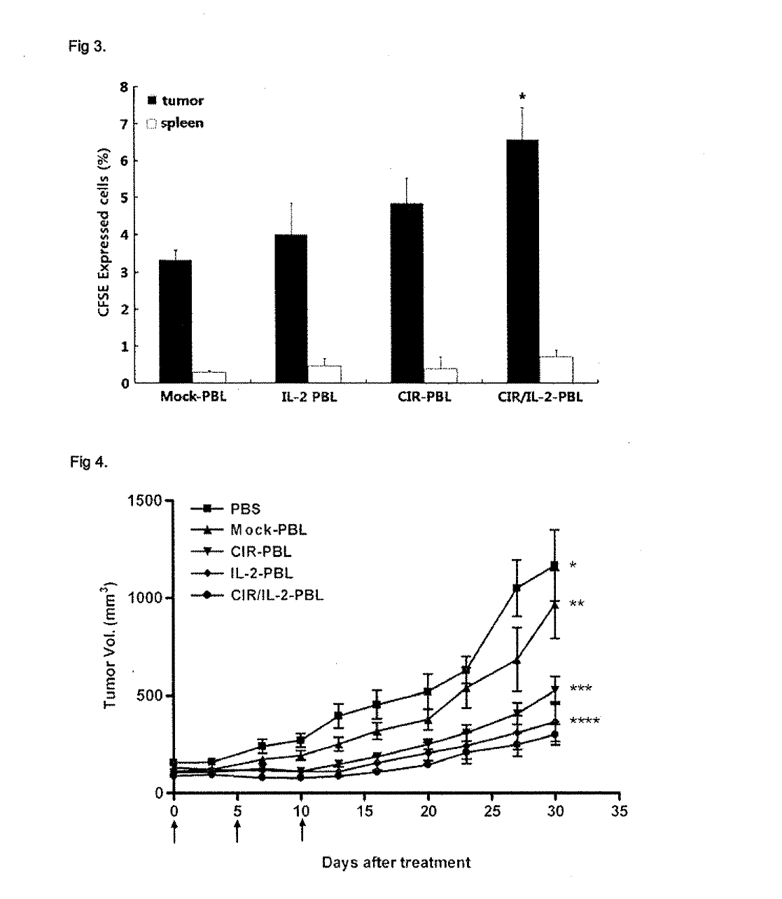

To investigate the localization of intra-tumorally administrated PBLs, we analyzed CFSE-labeled cells in the spleen and tumor by flow cytometry (FIG. 3). On day 1 after infusion, 6.56, 4.84, 4 and 3.33% CFSE-positive cells, respectively, were detected inside tumor tissue for CIR/IL-2-PBL, CIR-PBL, IL-2-PBL and mock-PBL. The frequency of CFSE positive cells in spleen was much lower than in tumor, with CIR/IL-2-PBL showing a slight increase compared with the other groups. These results suggest that trapping of infused PBLs in the tumor site by CIR is further enhanced by IL-2.

Antitumor Effects in Nude Mouse Model

A total of 1×10⁷ SKOV3 cells were subcutaneously injected into nude mice every 5 days for 15 days, at which time tumor volumes reached 50-100 mm³. Tumor-bearing mice that received CIR-PBL, IL-2-PBL or CIR/IL-2-PBL showed significantly slower tumor growth by day 30 after infusion than mice treated with PBS or mock-PBL (FIG. 4). Although tumors in mice treated with mock-PBL were smaller than those in the PBS control group mice, CIR/IL-2-PBL showed more inhibition of tumor growth than did mock-PBL (3.14-fold, **P=0.0068). Mice treated with CIR/IL-2-PBL or IL-2-PBL had smaller (2-, 1.7-fold) tumors compared with those of CIR-PBL (527 mm³). Interestingly, IL-2-PBL-treated tumors showed similar inhibition to those that received CIR/IL-2-PBL (317 vs 263 mm³; ****P=0.1230). These results show that transfer of IL-2 gene expression to CIR-PBL can enhance antitumor effects.

NK Cell Population in Tumor and Spleen

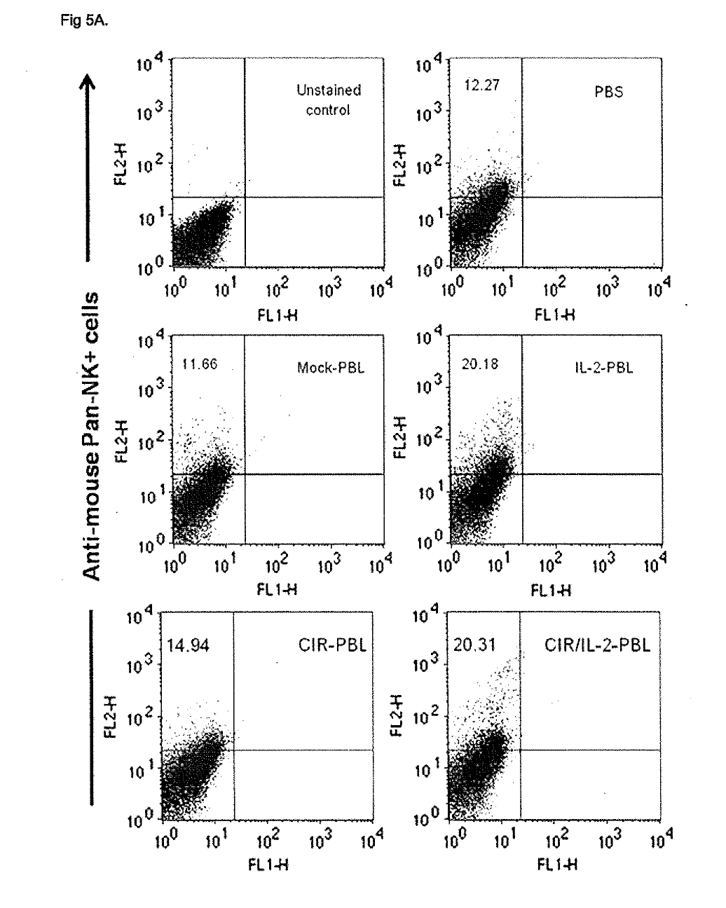

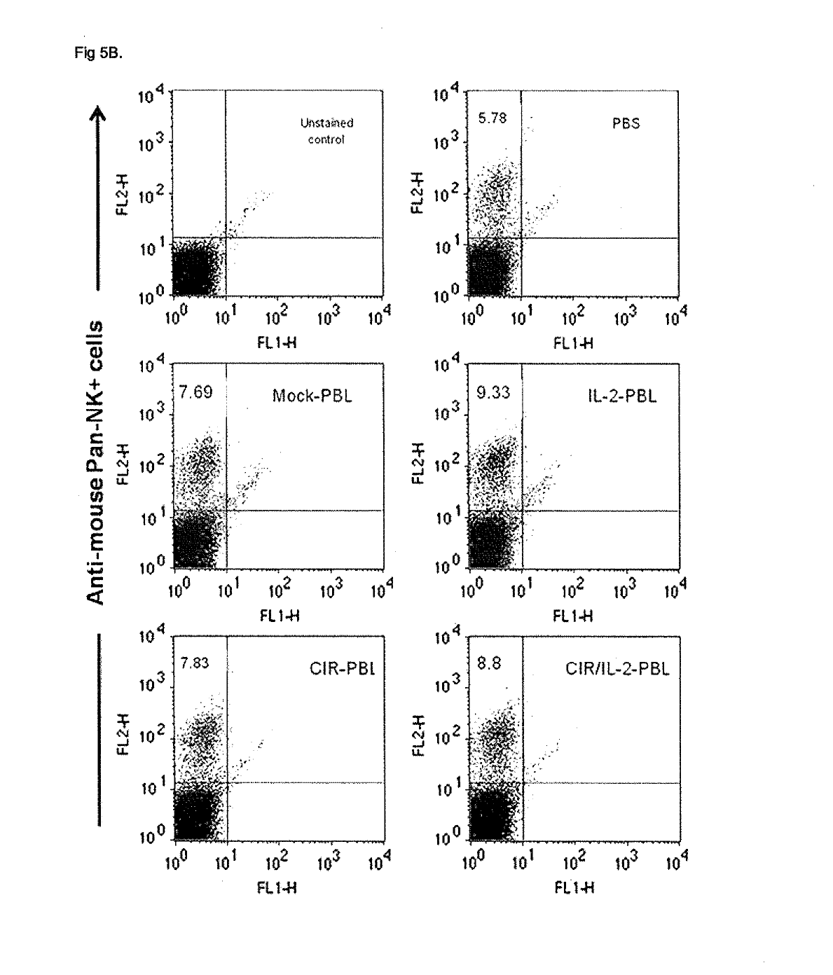

Because IL-2 is known to activate NK cells in addition to stimulating the proliferation of T cells, we measured the number of NK cells expressing a mouse Pan-NK marker, CD49b (DX5) in tumor site and spleen. The number of tumor-infiltrating NK cells was higher in the IL-2-PBLand CIR/IL-2-PBL-treated tumors (20.18±1.71 and 20.31±2.7%) than in the other groups (FIG. 5a). Although the number of NK cells in spleen was slightly increased in IL-2-PBL- and CIR/IL-2-PBL-treated mice compared with the other groups, the increase was not significant (FIG. 5b). These results suggest that IL-2 secreted from infused PBLs shows paracrine effects localized to the tumor site rather than systemic effects.

Antitumor Effects in NK Cell Depletion Model

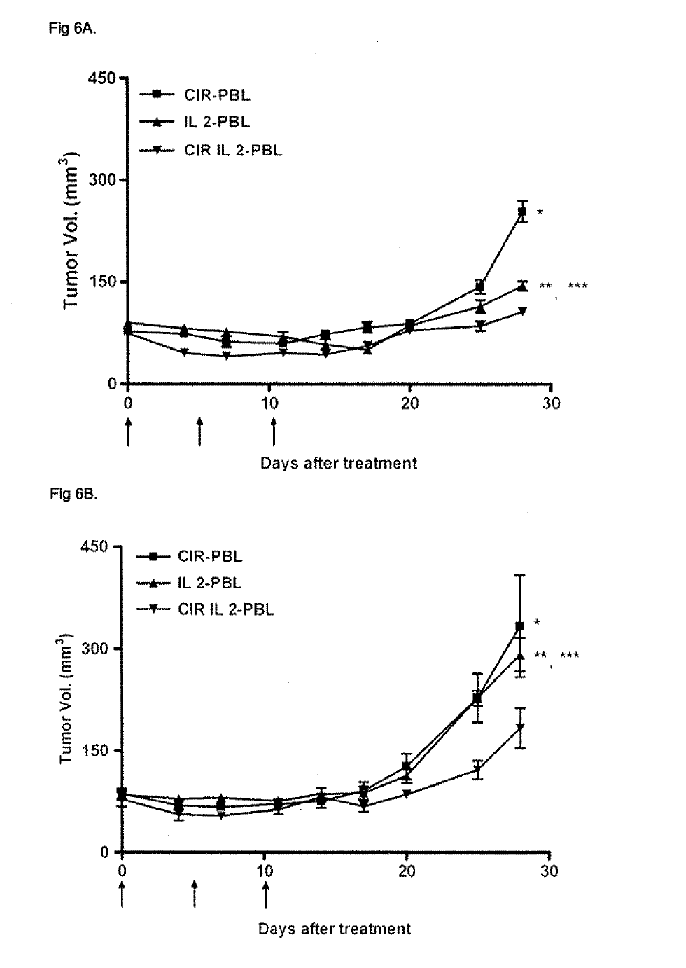

The strong inhibition of tumor growth by IL-2-PBL in vivo could be caused by activation of tumor-infiltrating NK cells by IL-2 and IFN-γ secreted from IL-2 transferred PBLs trapped in the tumor tissue. To test this hypothesis, we compared antitumor effects in the NK-cell-depleted model compared with the non-depleted one (FIG. 6), In the NK-cell-non-depleted model, IL-2-PBL and CIR/IL-2-PBL had significantly higher antitumor effects than CIR-PBL (1.42- and 1.63-fold, respectively); IL-2-PBL and CIR/IL-2-PBL had similar effects on tumor volume (287.5 and 250.3 mm3, **P=0.14). However, in the NK depletion model, IL-2-PBL had significantly lower antitumor effects than CIR/IL-2-PBL (1.54-fold, **P=0.045) and was similar to CIR-PBL. After treatment with IL-2-PBL in the presence of NK cell depletion, tumor volume decreased significantly compared with treatment in the absence of NK cell depletion (***P=0.01); thus, as a result of NK cell depletion, the antitumor effects of IL-2-PBL were decreased to a level similar to that effected by CIR-PBL. These results suggest that transfer of IL-2 RNA to CIR-PBL can activate NK cells to infiltrate around the tumor as well as prolong the survival of infused PBL in vivo.

An anticancer immunotherapeutic agent of the present invention can minimize systemic side effects due to a tumor site-specific antitumor effect, can prolong an anticancer effect due to the extended survival of a lymphocyte, and can be used to both prevent and treat cancer due to induction of an immune response by NK cells.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. An immunotherapeutic agent comprising a lymphocyte having an antigen-specific receptor gene and a bioactive material gene as an effective component.

2. The immunotherapeutic agent according to claim 1, wherein the antigen specific receptor is selected from the group consisting of a tumor antigen-specific receptor and an antigen-specific T cell receptor.

3. The immunotherapeutic agent according to claim 1, wherein the antigen-specific receptor is an anti-Her-2/neu-specific chimeric immune receptor.

4. The immunotherapeutic agent according to claim 1, wherein the bioactive material gene is selected from the group consisting of an IL-2 gene and a water-soluble TGF-beta receptor gene.

5. The immunotherapeutic agent according to claim 1, wherein the lymphocyte is a cell derived from a lymphocyte of peripheral blood, which is a T lymphocyte activated in a short term or a T-lymphocyte expanded on a large scale in vitro.

6. The immunotherapeutic agent according to claim 1, wherein the bioactive material gene is RNA of a bioactive material.

7. The immunotherapeutic agent according to claim 1, which is used to prevent or treat a tumor or a viral disease.

8. A method of preparing a lymphocyte, comprising:
providing an antigen-specific receptor gene and a bioactive material gene; and
transferring the genes to a lymphocyte.

9. The method according to claim 8, wherein the bioactive material gene is selected from the group consisting of an IL-2 gene and a water-soluble TGF-beta receptor gene.

10. The method according to claim 9, wherein the genes are RNAs.

11. The method according to claim 10, wherein the RNAs are transferred by electroporation or via liposomes.

12. A method of locally expressing a bioactive material in an antigen-specific lymphocyte-specific site using a lymphocyte to which genes of an antigen-specific receptor and a bioactive material are transferred.

13. The method according to claim 12, which is characterized by using an IL-2 gene as the bioactive material gene to prolong the survival time of the lymphocyte and to locally express the lymphocyte in an antigen-specific lymphocyte-specific site.

14. A method of preventing or treating a cancer or a viral disease comprising administrating a therapeutically effective amount of lymphocyte to which RNAs of a antigen-specific receptor and a bioactive material are introduced to a mammal.

15. The method according to claim 14, wherein the antigen specific receptor is selected from the group consisting of a tumor antigen-specific receptor and an antigen-specific T cell receptor.

16. The method according to claim 14, wherein the antigen-specific receptor is an anti-Her-2/neu-specific chimeric immune receptor.

17. The method according to claim 14, wherein the bioactive material gene is selected from the group consisting of an IL-2 gene and a water-soluble TGF-beta receptor gene.

18. The method according to claim 14, wherein the lymphocyte is a cell derived from a lymphocyte of peripheral blood, which is a T lymphocyte activated in a short term or a T-lymphocyte expanded on a large scale in vitro.

19. The method according to claim 14, wherein the bioactive material gene is RNA of a bioactive material.
